(19) United States
(12) Patent Application Publication
(10) Pub. No.: US 2023/0210816 A1
(43) Pub. Date: Jul. 6, 2023

(54) INHIBITION OF ARENAVIRUSES BY COMBINATIONS OF APPROVED THERAPEUTIC DRUGS

(71) Applicants: UNIVERSITY OF WASHINGTON, Seattle, WA (US); UNIVERSITY OF VIRGINIA PATENT FOUNDATION, Charlottesville, VA (US)

(72) Inventors: Stephen J. Polyak, Seattle, WA (US); Shawn Herring, Seattle, WA (US); Jessica Wagoner, Seattle, WA (US); Judith White, Charlottesville, VA (US)

(73) Assignees: UNIVERSITY OF WASHINGTON, Seattle, WA (US); UNIVERSITY OF VIRGINIA PATENT FOUNDATION, Charlottesville, VA (US)

(21) Appl. No.: 17/998,800

(22) PCT Filed: May 24, 2021
(86) PCT No.: PCT/US2021/033865

§ 371 Date: Nov. 14, 2022

Related U.S. Application Data
(60) Provisional application No. 63/030,764, filed on May 27, 2020.

(51) Int. Cl. A61K 31/4045; A61K 31/496; A61K 31/47; A61K 31/135; A61K 9/00; A61P 31/14

(52) U.S. Cl. CPC A61K 31/4045; A61K 31/496; A61K 31/47; A61K 31/135; A61K 9/0053; A61P 31/14

(57) ABSTRACT

Methods for inhibiting or treating viral infection in a subject infected with a vims of the arenaviridae family with a therapeutic agent combination: (a) arbidol and aripiprazole; (b) arbidol and amodiaquine; (c) arbidol and sertraline; (d) arbidol, iprazole, and amodiaquine; (e) arbidol, aripiprazole, and sertraline; or (f) aripiprazole and amodiaquine; or pharmaceutically acceptable salts thereof.

### CROSS-REFERENCE TO RELATED APPLICATION

This application claims the benefit of U.S. Application No. 63/030,764, filed May 27, 2020, expressly incorporated herein by reference in its entirety.

### STATEMENT OF GOVERNMENT LICENSE RIGHTS

This invention was made with government support under Grant No. AI114776, awarded by the National Institutes of Health. The government has certain rights in the invention.

### BACKGROUND OF THE INVENTION

Members of Arenaviridae, a family of enveloped viruses with segmented RNA genome, are capable of causing hemorrhagic fevers and neurological diseases. Among its three genera, mammarenaviruses are known to cause disease in humans. Mammarenaviruses are further classified into Old World (OW) and New World (NW) arenaviruses with outbreaks occurring respectively in Africa and the Americas. There are at least 15 OW arenaviruses. Examples of OW arenaviruses include Lassa Virus (LASV), Lymphocytic Choriomeningitis Virus (LCMV) and Mopeia virus. LASV is estimated to cause about 100,000 to 300,000 human infections and about 5,000 human deaths per year. LCMV, which is deleterious in pregnant women and transplant recipients, is present globally. There are over 20 different NW arenaviruses including Junin (JUNV), Pichinde (PICV), Chapare (CHAPV) and Tacaribe (TACV) viruses. JUNV has caused outbreaks of hemorrhagic fever in Argentina with an estimated case fatality rate of 15-30%. Although an effective vaccine has been developed, JUNV infections are generally treated with supportive care and in some cases convalescent serum. Aside from those countermeasures and the use of ribavirin for severe cases of LASV, there are no approved vaccines, therapeutic antibodies or drugs with which to treat patients infected with an arenavirus.

Drug screens have identified small molecules with activity against LASV and other arenaviruses. Most are investigational drugs and include clotrimazole derivatives, ST-193, F3406, LHF-535, kinase inhibitors, losmapimod, and inhibitors of purine and pyrimidine biosynthesis. The polymerase inhibitors remdesivir and favipiravir have shown some activity against arenaviruses in cell cultures and in vivo, respectively. Approved drugs that surfaced in arenavirus drug screens were mycophenolic acid, a broad-spectrum inhibitor of purine biosynthesis; leflunomide, an inhibitor of pyrimidine biosynthesis; the calcium channel blockers lacidipine, nifedipine, and verapamil; and gabapentin. Several of these drugs (e.g., ST-193 and F3406) block the entry stage, while others (e.g., remdesivir and the purine and pyrimidine synthesis inhibitors) block the replication stage of the arenavirus lifecycle.

Despite advances in the treatment of viral infections, a need exists for improved methods of treatment and improved compositions for treating viral infections. The present invention seeks to fulfill this need and provides further related advantages.

### SUMMARY OF THE INVENTION

In one aspect, the present invention provides methods for inhibiting or treating viral infection in a subject infected with a virus of the arenaviridae family.

In certain embodiments, the therapeutic agent combination is a combination of one of (a) arbidol and aripiprazole; (b) arbidol and amodiaquine; (c) arbidol and sertraline; (d) arbidol, aripiprazole, and amodiaquine; (e) arbidol, aripiprazole, and sertraline; or (f) aripiprazole and amodiaquine; or pharmaceutically acceptable salts thereof.

In other embodiments, the therapeutic agent combination comprises arbidol, or a pharmaceutically acceptable salt thereof, and a second therapeutic agent or a combination of second therapeutic agents selected from (a) aripiprazole or a pharmaceutically acceptable salt thereof; (b) amodiaquine or a pharmaceutically acceptable salt thereof; (c) sertraline or a pharmaceutically acceptable salt thereof; (d) aripiprazole and amodiaquine, or pharmaceutically acceptable salts thereof; or (e) aripiprazole and sertraline, or pharmaceutically acceptable salts thereof.

In another aspect, pharmaceutical compositions of the therapeutic agent combinations are provided.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIGS. 1A-1C illustrates that arbidol inhibits pseudoviruses expressing multiple arenavirus glycoproteins: JUNV (FIG. 1A); LCMV (FIG. 1B); and

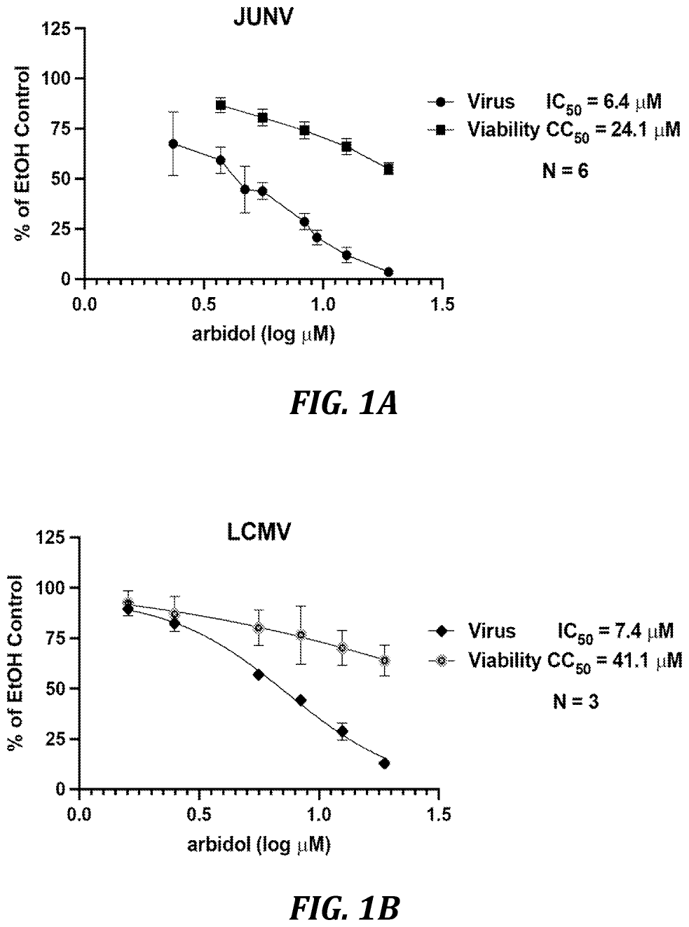

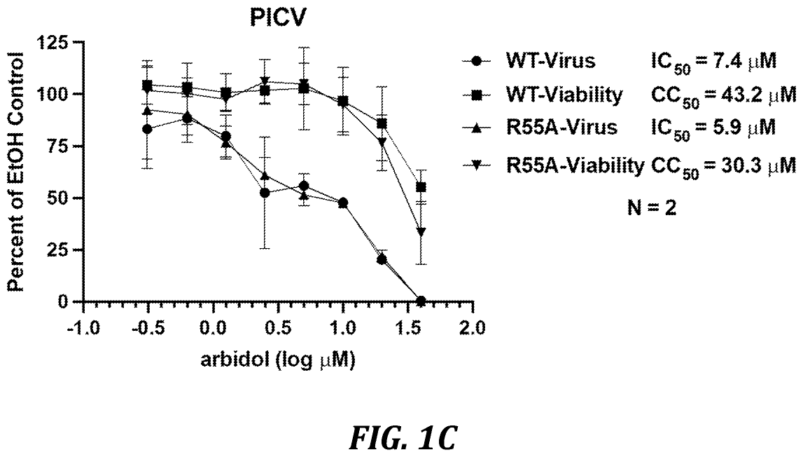

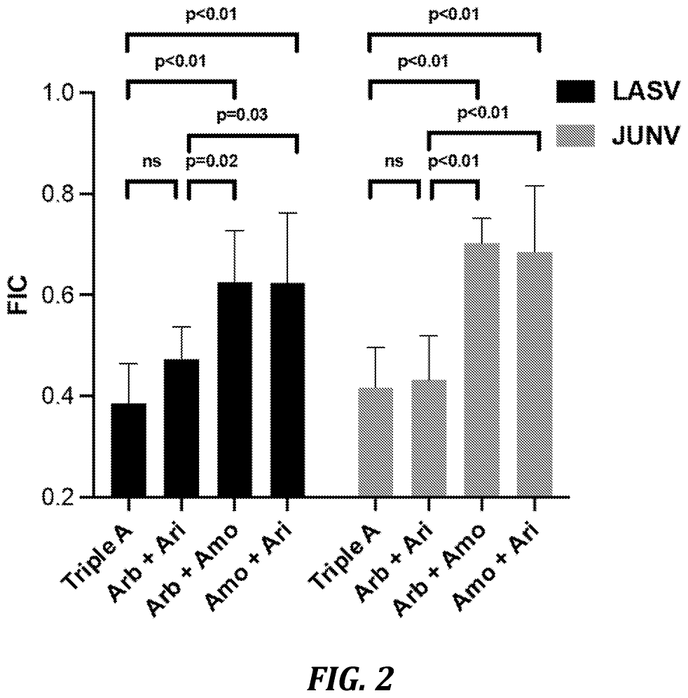

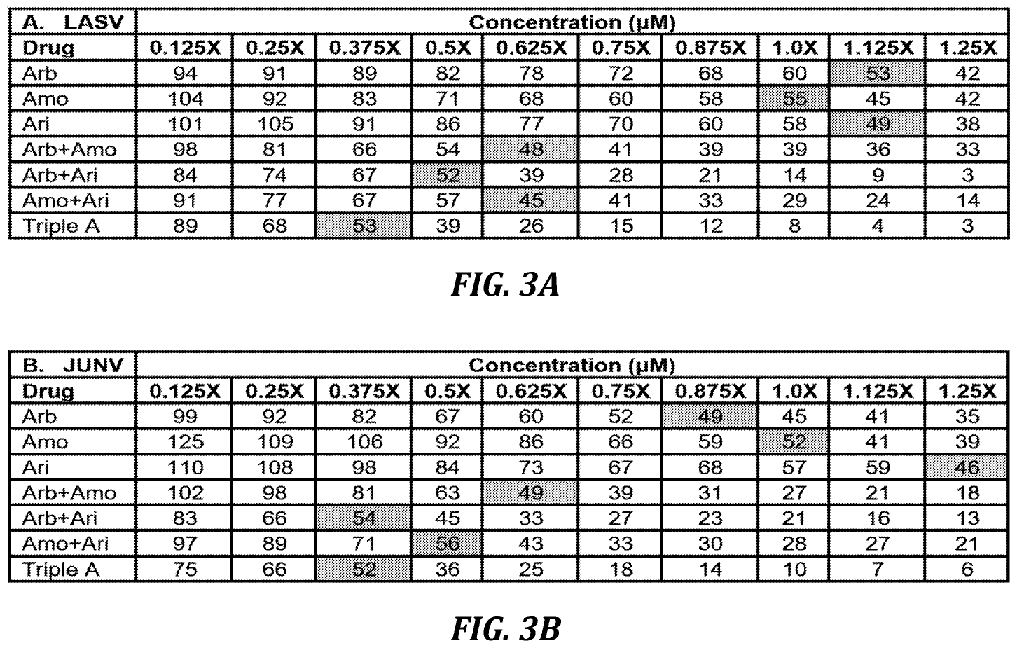

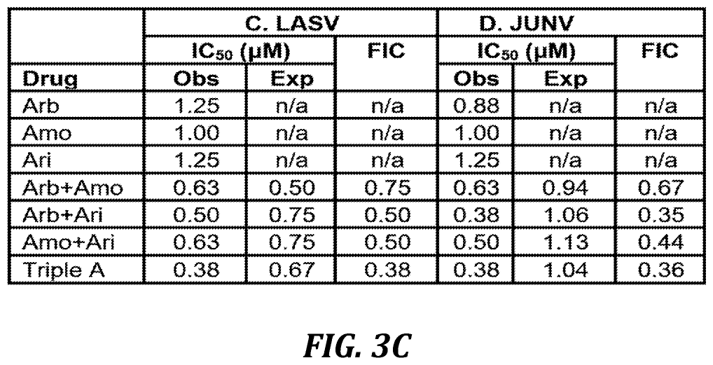

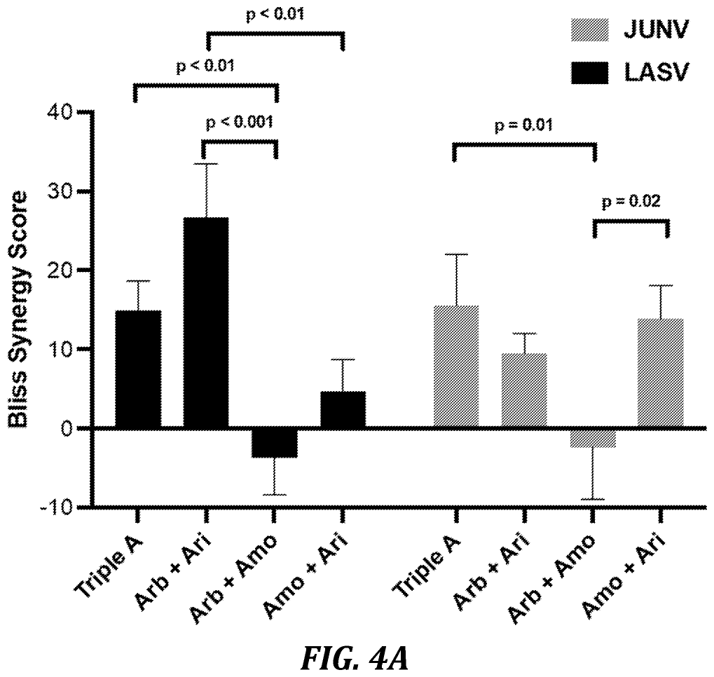

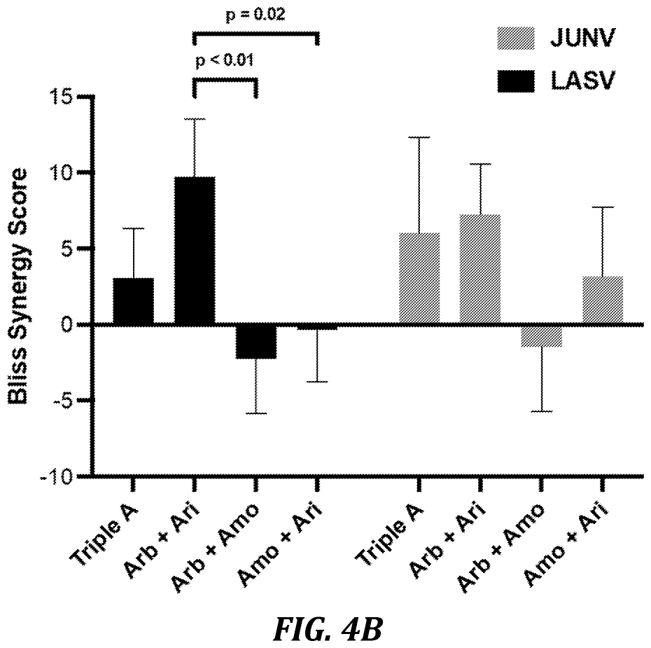

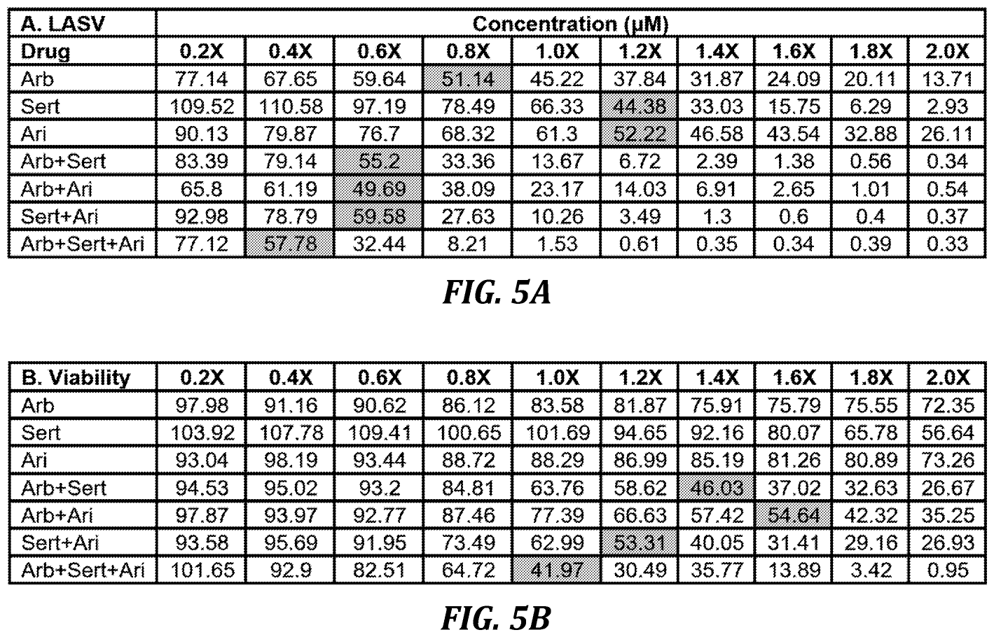

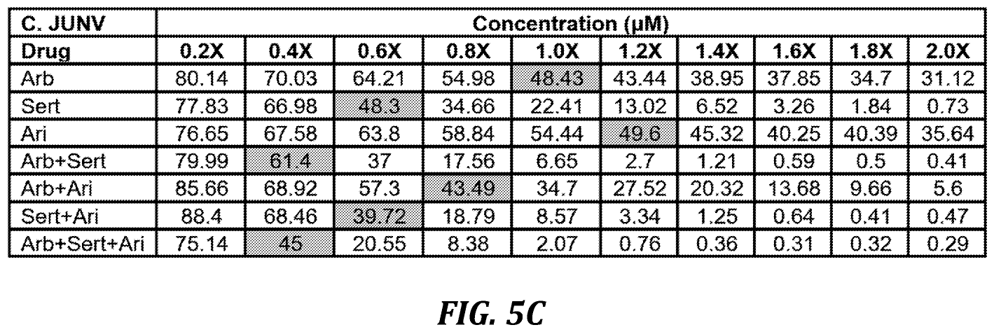

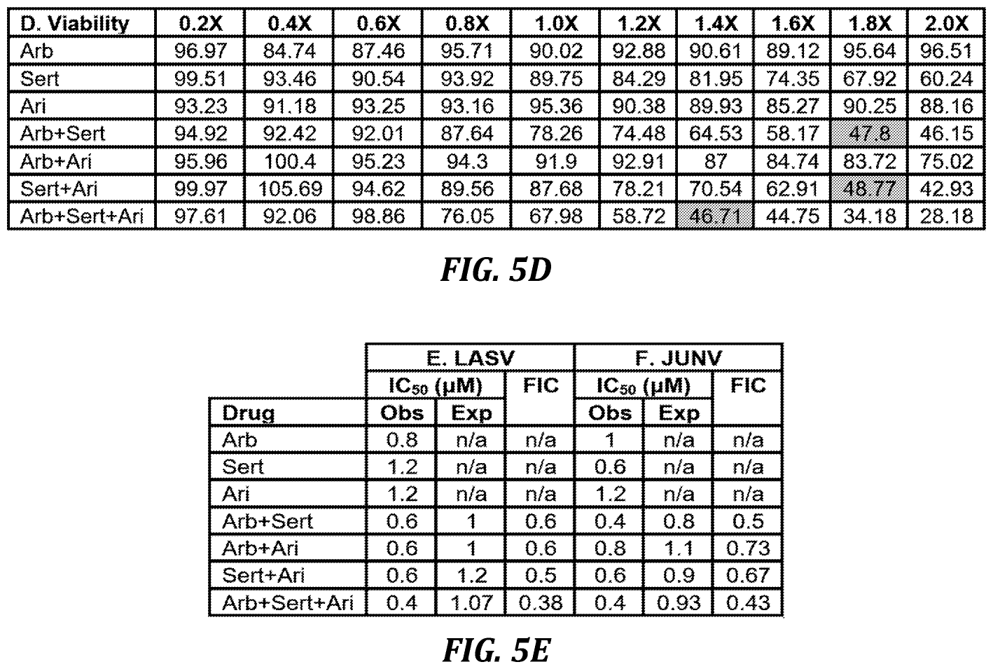

PICV (FIG. 1C). Vero cells were treated with varying concentrations of arbidol prior to infection with MLV pseudoviruses that enter cells via the glycoproteins of JUNV, LCMV, PICV WT or PICV R55A. Twenty-four hours later, luciferase activity was measured to quantify virus infection, and ATP levels were measured to quantify cell viability. Error bars represent standard deviations. Each condition was performed in triplicate, and each experiment was performed 6, 3, 2 and 2 times for JUNV, LCMV, PICV WT, and PICV R55A. For each virus, the data depict the averages and standard deviations across all experiments performed for that virus. IC₅₀ and CC₅₀ were calculated in Prism.

FIG. 2 compares synergistic suppression of LASV and JUNV glycoprotein-bearing pseudoviruses by arbidol combined with aripiprazole. Vero cells were treated with varying concentrations of a single drug or a two- or three-drug combination of arbidol (Arb), amodiaquine (Amo), and aripiprazole (Ari) before infection with LASV or JUNV pseudovirus. Triple A refers to the combination of the three drugs. Twenty-four hours post-infection, luciferase activity was measured. To quantify the combination effects for each experiment, the Fractional Inhibitory Concentration (FIC) score was calculated by dividing the observed IC₅₀ by the expected IC₅₀ for each combination. For LASV (dark bars), data represent averages and standard deviations from 8 separate experiments, with Z scores of >0.6 (average Z score was 0.71+/−0.06) and where each condition was conducted in triplicate. The 1× concentrations of Arb, Amo, and Ari were 11, 8, and 12 μM, respectively. For JUNV (lighter bars), data represent averages and standard deviations from 6 separate experiments, with Z scores of >0.2 (the average Z score was 0.5+/−0.11), and where each condition was conducted in triplicate. The 1× concentrations of Arb, Amo, and Ari were 8, 12, and 5 μM, respectively. FICs <1 suggest synergy. P values are derived from one-way ANOVA using Tukey's multiple comparisons test in GraphPad Prism; ns=not significant.

FIGS. 3A-3C are representative plate maps of synergistic combination testing against LASV and JUNV pseudoviruses. Vero cells were treated with the indicated concentrations of a single drug or a two- or three-drug combination of arbidol (Arb), amodiaquine (Amo), and aripiprazole (Ari) before infection with LASV (FIG. 3A) or JUNV pseudovirus (FIG. 3B). Triple A refers to the combination of the three drugs. Data are expressed as percent of infected cells treated with solvent control. Twenty-four hours post-infection, luciferase activity was measured. The concentration of each drug alone providing about 50% inhibition of infection, the concentration of each drug needed in the pairwise combinations to produce about 50% inhibition of infection, and the concentration of each drug needed in the three-drug cocktail to yield about 50% inhibition of infection is shown (see shading). The data show that the concentration of each drug required to inhibit LASV and JUNV pseudovirus infection shifts to the left (i.e., decreases) from a single drug, to the two-drug, and to three-drug combinations. Summary FIC scores for the drugs against LASV (FIG. 3C, panel C) and JUNV (FIG. 3C, panel D) are shown. Z-Factors for each assay were 0.76 and 0.56, respectively, and the signal to noise ratios were 22,546 and 34,059 for LASV and JUNV, respectively. For LASV, the 1× concentrations of Arb, Amo, and Ari were 11, 8, and 12 μM, respectively. For JUNV, the 1× concentrations of Arb, Amo, and Ari were 8, 12, and 5 μM, respectively. Obs=observed; Exp=expected; n/a=not applicable; FIC=fractional inhibitory concentration.

FIGS. 4A and 4B illustrates the synergy of arbidol with aripiprazole to suppress arenaviruses. All data were analyzed by SynergyFinder2. Error bars represent the averages of the standard deviations of the overall Bliss Synergy Scores. FIG. 4A, representative pseudovirus results. For LASV (dark bars), data are derived from a single experiment with a Z score of 0.74 and a signal to noise ratio of 33139. The 1× concentrations of Arb, Amo, and Ari were 11, 8, and 12 μM, respectively. For JUNV (lighter bars), data are derived from a single experiment with a Z score of 0.29 and a signal to noise ratio of 34193. The 1× concentrations of Arb, Amo, and Ari were 8, 12, and 5 μM, respectively. FIG. 4B, data reflect the composite from 8 and 6 separate experiments performed using Drug Combination Assay 1 for LASV and JUNV, respectively. Indicated p values are derived from one-way ANOVA using Tukey's multiple comparisons test in GraphPad Prism. P-values for all other comparisons were >0.05. Data represent averages and standard deviations of triplicate conditions for each drug combination.

FIGS. 5A-5E tabulate synergistic suppression of LASV and JUNV GP-bearing pseudoviruses by arbidol combined with the approved drugs aripiprazole and sertraline. Vero cells were treated with varying concentrations of single drug or a two- or three-drug combination of arbidol (Arb), sertraline (Sert), and aripiprazole (Ari) before infection with LASV or JUNV pseudovirus. Data are expressed as percent of infected cells treated with solvent control. Twenty-four hours post-infection, virus infection (FIGS. 5A and 5C) and viability was measured (FIGS. 5B and 5D). In FIGS. 5A and 5C, the concentration of each drug alone, the concentration of each drug needed in the pairwise combinations to produce 50% inhibition of infection, and the concentration of each drug needed in the three-drug cocktail to yield about 50% inhibition of infection is shown (see shading). FIGS. 5B and 5D show the drug concentrations that inhibit cell viability by approximately 50%. Summary FIC scores for the drugs against LASV (FIG. 5E, panel E) and JUNV (FIG. 5E, panel F) are shown. The data are from a single experiment where each condition was conducted in triplicate. For both viruses, the 1× concentrations of Arb, Sert, and Ari were 9 μM. For LASV and JUNV, Z-Factors for each assay were 0.71 and 0.52, respectively, while the signal to noise vales were 27,385 and 335 for LASV and JUNV, respectively. Obs=observed; Exp=expected; n/a=not applicable; FIC=fractional inhibitory concentration.

### DETAILED DESCRIPTION OF THE INVENTION

The present invention provides methods for inhibiting or treating viral infection in a subject infected with a virus of the arenaviridae family with therapeutic agent combinations: (a) arbidol and aripiprazole; (b) arbidol and amodiaquine; (c) arbidol and sertraline; (d) arbidol, aripiprazole, and amodiaquine; (e) arbidol, aripiprazole, and sertraline; or (f) aripiprazole and amodiaquine; or pharmaceutically acceptable salts thereof. Compositions of the therapeutic agent combinations are also provided.

In one aspect, the invention provides methods for inhibiting or treating viral infection in a subject infected with a virus of the arenaviridae family. In the methods, a therapeutically effective amount of a therapeutic agent combination is administered administering to a subject in need thereof.

In certain embodiments, the therapeutic agent combination is one selected from:

(a) arbidol and aripiprazole, or pharmaceutically acceptable salts thereof;

(b) arbidol and amodiaquine, or pharmaceutically acceptable salts thereof;

(c) arbidol and sertraline, or pharmaceutically acceptable salts thereof;

(d) arbidol, aripiprazole, and amodiaquine, or pharmaceutically acceptable salts thereof;

(e) arbidol, aripiprazole, and sertraline, or pharmaceutically acceptable salts thereof; and

(f) aripiprazole and amodiaquine, or pharmaceutically acceptable salts thereof.

In other embodiments, the therapeutic agent combination comprises arbidol, or a pharmaceutically acceptable salt thereof, and a second therapeutic agent or a combination of second therapeutic agents. In certain of these embodiments, the second therapeutic agent or combination of second therapeutic agents is one selected from:

(a) aripiprazole or a pharmaceutically acceptable salt thereof;

(b) amodiaquine or a pharmaceutically acceptable salt thereof;

(c) sertraline or a pharmaceutically acceptable salt thereof;

(d) aripiprazole and amodiaquine, or pharmaceutically acceptable salts thereof; and

(e) aripiprazole and sertraline, or pharmaceutically acceptable salts thereof. In one embodiment, the therapeutic agent combination comprises arbidol and aripiprazole, or pharmaceutically acceptable salts thereof.

In another embodiment, the therapeutic agent combination comprises arbidol and amodiaquine, or pharmaceutically acceptable salts thereof.

In a further embodiment, the therapeutic agent combination comprises arbidol and sertaline, or pharmaceutically acceptable salts thereof.

In another embodiment, the therapeutic agent combination comprises arbidol, aripiprazole, and amodiaquine, or pharmaceutically acceptable salts thereof.

In a further embodiment, the therapeutic agent combination comprises arbidol, aripiprazole, and sertraline, or pharmaceutically acceptable salts thereof.

In another embodiment, the therapeutic agent combination comprises aripiprazole and amodiaquine, or pharmaceutically acceptable salts thereof.

In the methods of the invention, a subject infected with a virus of the arenaviridae family is administered a therapeutic agent combination as described herein. In addition to the specific viruses of the arenaviridae family described herein, the methods are effective to treat old and new world arenaviruses, as well as emerging arenaviruses. In general, the methods of the invention are effective to treat arenaviruses that infect cells by endosomal entry as well as viruses whose genetic sequence place such viruses in the arenavirus family.

The methods of the invention are effective to treat old world arenaviruses including LCM, Lassa, Mopeia, Mobala, Ippy, Merino Walk, Menekre, Gbagroube, Morogoro, Kodoko, Lujo, Lemniscomys, Mus minutoides, and Luna viruses. The methods of the invention are effective to treat new world arenaviruses including Tacaribe, Junin, Machupo, Cupixi, Amapari, Parana, Tamiami, Pichinde, Latino, Flexal, Guanarito, Sabia, Oliveros, Whitewater Arroyo, Pirital, Pampa, Bear Canyon, Ocozocoautla de Espinosa, Allpahuayo, Tonto Creek, Big Brushy Tank, Real de Catorce, Catarina, Skinner Tank, and Chapare viruses.

In certain embodiments, the method is effective to inhibit or treat viral infections caused by arenaviruses including Lassa virus, Junin virus, Guanarito virus, Lujo virus, Machupo virus, Sabia virus, and Whitewater Arroyo virus.

As used herein “therapeutically effective amount” refers to an amount of the therapeutic drug combination administered to the subject that results in an alleviation of the viral infection (e.g., an improvement in the condition of the subject so treated). The therapeutically effective amount will vary for each therapeutic drug combination and for each therapeutic agent in the combination, as well as from subject to subject based on the subject's condition (e.g., the severity of the infection and how long the subject has been infected).

In certain embodiments, the therapeutically effective amount of the therapeutic agent combination is a synergistic amount of each therapeutic agent in the combination, as described herein.

In certain embodiments, the therapeutic agent combination is administered as a single composition. In other embodiments, the therapeutic agent combination is administered as separate individual therapeutic agents.

In certain embodiments of the methods, the therapeutic agent combination is orally administered.

In another aspect, the invention provides pharmaceutical compositions useful in the methods of the invention.

In one embodiment, the pharmaceutical composition comprises arbidol and aripiprazole, or pharmaceutically acceptable salts thereof.

In another embodiment, the pharmaceutical composition comprises arbidol and amodiaquine, or pharmaceutically acceptable salts thereof.

In a further embodiment, the pharmaceutical composition comprises arbidol and sertraline, or pharmaceutically acceptable salts thereof.

In another embodiment, the pharmaceutical composition comprises aripiprazole and amodiaquine, or pharmaceutically acceptable salts thereof.

In a further embodiment, the pharmaceutical composition comprises arbidol, aripiprazole, and amodiaquine, or pharmaceutically acceptable salts thereof.

In another embodiment, the pharmaceutical composition comprises arbidol, aripiprazole, and sertraline, or pharmaceutically acceptable salts thereof.

In certain embodiments, the pharmaceutical compositions of the invention further include a pharmaceutically acceptable carrier. Suitable pharmaceutically acceptable carriers include diluents, excipients, liposomes, polymeric micelles, microspheres, nanoparticles that are useful for administering the individual therapeutic agents, which are known and approved therapeutic agents, of the therapeutic drug combinations described herein.

In certain embodiments. the pharmaceutical compositions of the invention are formulated for oral administration. Suitable formulations include those useful for administering the individual therapeutic agents of the therapeutic drug combinations described herein (e.g., solid forms, such as tablets and capsules, or liquid forms including suspensions or syrups).

In certain embodiments. the pharmaceutical compositions of the invention include a synergistic amount of each therapeutic agent of the therapeutic drug combination.

The present invention provides synergistic combinations of approved drugs for use at the inception of new viral outbreaks. The concept is that once the family of the causative virus is identified through genomic sequencing, in this instance/application, any other virus that is placed within the arenavirus family, or is currently designated as an arenavirus, there would be a shelf-ready cocktail of approved drugs for immediate use. A cocktail documented in advance to reduce titers by multiple members of the implicated virus family would be highly beneficial. Approved drugs have many positive features for this purpose, including shelf-ready availability, relative low cost, room temperature stability, delivery by the oral route, utility in non-hospitalized settings, and known pharmacology. The approach employs combinations of approved drugs, as a frequent limitation of monotherapy with a drug approved for another indication is the inability to achieve viral suppression (reflected in the concentration of the drug that suppresses a virus by 50% (IC₅₀)) at concentrations that are clinically achievable. With synergistic drug combinations, the dose of the individual drugs needed for anti-viral activity is lowered, thereby allowing the maximum serum concentration (Cₘₐₓ) to approach, equal, or exceed the IC₅₀ while reducing the risk of adverse effects. Other advantages of drug combination approaches include possible reductions in development of viral resistance.

Towards the goal of developing combinations of approved drugs against (re)emerging viral infections, combinations of approved drugs that synergistically suppress EBOV in cell cultures have been identified. The emphasis in those studies was on drugs that block virus entry, the first step in all viral lifecycles. To begin to broaden this approach, the methods of the invention further identified approved drugs with activity against both EBOV (a filovirus) and LASV (an OW arenavirus). While EBOV and LASV utilize different cell surface attachment factors and endosomal receptors, they share important features in their fusion and entry mechanisms: (i) both viruses display multiple copies of a trimeric class I fusion glycoprotein termed GP; (ii) each trimer is composed of heterodimers combining a receptor binding subunit and a fusion subunit, the latter containing an internal fusion loop; (iii) the viruses are taken into cells by macropinocytosis; (iv) the viral GPs are triggered by a combination of binding to an endosomal receptor and exposure to low endosomal pH which convert the GPs to their fusogenic, trimer-of-hairpins conformation.

Reflecting these shared traits and features of virus entry, several approved drugs suppress the entry into cells of both EBOV and LASV. As described herein, combination testing of arbidol, a fusion blocker, with several other approved drugs against LASV and JUNV (another arenavirus) GP-bearing pseudoviruses was studied. Arbidol, which inhibits FLU entry by inactivating the viral hemagglutinin (HA) protein, also inhibits hepatitis C virus, EBOV, LASV, JUNV, Tacaribe virus (TACV), Zika virus, and SARS-CoV-2 perhaps through a similar mechanism. Drugs previously shown to inhibit EBOV and/or LASV at discrete steps of the virus entry pathway were also evaluated: aripiprazole (a mood stabilizing drug that blocks macropinocytotic internalization), amodiaquine (a lysosomotropic antimalarial drug that increases endosome pH), sertraline (an antidepressant that blocks fusion), and niclosamide (an antihelminthic drug that inhibits entry of several viruses). In addition to blocking EBOV, LASV and TACV, as described herein, arbidol blocks entry mediated by the GPs of other arenaviruses including LCMV, JUNV, and PICV, and MARV filoviruses. Moreover, arbidol, amodiaquine, aripiprazole, sertraline, and niclosamide also inhibit infection of cells by infectious PICV, and arbidol, sertraline and niclosamide also inhibit fully infectious LASV. Combinations of [arbidol plus aripiprazole] and [arbidol plus sertraline] are demonstrated to synergistically suppress infections mediated by the GPs of LASV and JUNV.

Arbidol Inhibits Multiple Arenaviruses

Arbidol inhibits infection mediated by the LASV and EBOV GPs. Arbidol was tested to determine whether it inhibits infection mediated by the GPs of other arenaviruses and filoviruses. MLV luciferase reporter viruses pseudotyped with LASV, JUNV, LCMV, EBOV, MARV Angola, and MARV Musoke GPs were generated, and expression of the relevant GP was confirmed in pseudovirus stocks. Arbidol inhibited infection of Vero cells with MLV reporter viruses pseudotyped with GPs from the arenaviruses JUNV, LCMV, and PICV GPs (see FIGS. 1A-1C, respectively). For PICV, pseudoviruses carrying wild type (WT) and a mutant (R55A) PICV GP, which was previously shown to have enhanced fusion activity, were tested. Arbidol inhibited infection of cells with all four pseudoviruses, with IC₅₀s of 6.4, 7.4, 7.4, and 5.9 μM for JUNV, LCMV, PICV WT, and PICV R55A (see FIGS. 1A-1C, respectively). The selectivity indices (CC₅₀/IC₅₀) were 3.8, 5.6, 5.8, and 5.1, respectively. Arbidol also inhibited MLV pseudoviruses bearing filovirus glycoproteins from MARV Angola and MARV Musoke, with IC₅₀s of 3.9, and 3.5 μM and selectivity indices of 13.2 and 10.6, respectively.

Approved Drugs Inhibit Infectious PICV and LASV

Studies of arbidol and the approved drugs amodiaquine, aripiprazole, sertraline, and niclosamide we extended versus arenaviruses using Biosafety Level 2 (BSL2)-compatible infectious PICV containing a green fluorescent protein (GFP) reporter (see Dhanwani R, Zhou Y, Huang Q, Verma V, Dileepan M, Ly H, Liang Y. 2015. A Novel Live Pichinde Virus-Based Vaccine Vector Induces Enhanced Humoral and Cellular Immunity after a Booster Dose. J Virol 90:2551-60). The PICV-GFP virus is a fully replication-competent arenavirus that encodes all WT viral proteins and RNA elements and utilizes the same mechanism of replication as the WT PICV, including the PICV GPC-mediated cell entry, the L and NP-dependent viral RNA replication and transcription, and the matrix protein Z-mediated virus assembly and budding. All five drugs inhibited PICV-GFP infection of Vero E6 cells with IC₅₀s of 8.4, 4.5, 5.4, 3.7, and <0.2 μM for arbidol, amodiaquine, aripiprazole, sertraline, and niclosamide, respectively (See FIGS. 2 and 3A-3C). Moreover, arbidol, sertraline, and niclosamide inhibited infectious LASV with similar IC₅₀s of 10, 7, and 0.2 μM, respectively (See FIGS. 3A-3C). Collectively, the data indicate that arbidol, amodiaquine, aripiprazole, sertraline, and niclosamide inhibit multiple arenaviruses.

Arbidol Synergizes with Other Approved Drugs to Suppress Arenavirus GP-Mediated Infection

Combinations of arbidol (fusion inhibitor), amodiaquine (endosomal acidification inhibitor), and aripiprazole (virus internalization inhibitor) were first evaluated using Drug Combination Assay 1 (see Materials and Methods). Average fractional inhibitory concentration (FIC) data was determined for the two- and three-drug combinations against LASV-GP and JUNV-GP pseudovirus infections of Vero cells. FIC is a ratio of the observed IC₅₀ to the expected IC₅₀ for each two-drug and three-drug combination. The expected IC₅₀ for the two- or three-drug combinations is the arithmetic mean of the IC₅₀ for each drug tested individually. By examining the single drug dose-response data, the concentration closest to 50% inhibition was designated as the observed IC₅₀. FICs of 1 suggest additivity, FICs >1 suggest antagonism, and FICs <1 suggest synergism. All drug combinations yielded FICs <1, suggesting synergy. The triple-drug combination of [arbidol+amodiaquine+aripiprazole] (referred to as “Triple A”) and the two-drug combination of [arbidol+aripiprazole] produced the lowest and similar FICs for suppression of LASV and JUNV infection. Moreover, the Triple A combination and the two-drug combination of [arbidol+aripiprazole] produced FICs that were statistically significantly lower (suggesting greatest synergy) than those for the other two-drug combinations [arbidol+amodiaquine] and [amodiaquine+aripiprazole]. When viewed from the perspective of the plate map, two- and three-drug combinations required lower concentrations to achieve approximate IC₅₀ suppression of virus infection compared to the single drugs, and drug combinations yielded FICs <1, suggesting synergy (see FIG. 3C, panels C and D). Thus, the two- and three-drug combinations lowered the IC₅₀ for inhibition of LASV and JUNV pseudoviruses. For example, using the formula for percent reduction [(monotherapy concentration−combination concentration)/monotherapy concentration)×100], the Triple A combination and the two-drug combination of [arbidol+aripiprazole] yielded approximately 70% reductions in the concentrations of arbidol and aripiprazole needed for LASV and JUNV suppression.

SynergyFinder2 (Ianevski A, Giri A K, Aittokallio T. 2020. SynergyFinder 2.0: visual analytics of multi-drug combination synergies. Nucleic Acids Res doi:10.1093/nar/gkaa216; and Ianevski A, He L, Aittokallio T, Tang J. 2017. SynergyFinder: a web application for analyzing drug combination dose-response matrix data. Bioinformatics 33:2413-2415) was then used to further analyze results from these LASV and JUNV experiments. In individual experiments, the Triple A and [arbidol+aripiprazole] combinations were among the most synergistic combinations for both LASV and JUNV, as evidenced by overall Bliss synergy scores >10 (FIG. 4A). When averaged across all eight experiments, [arbidol+aripiprazole] synergistically inhibited LASV, as evidenced by a Bliss Synergy score that was significantly higher than those of the other two, two-drug combinations (FIG. 4B). For JUNV, the Triple A combination and the combination of [arbidol+aripiprazole] showed a trend towards additive to synergistic suppression of pseudovirus infection across all six experiments (FIG. 4B). The combinations of [arbidol+amodiaquine] and [amodiaquine+aripiprazole] did not confer significant synergy, consistent with the higher FICs produced by this drug combination. These data suggest that for the three drugs tested, the two-drug combination of [arbidol+aripiprazole] caused synergistic suppression of arenaviruses. Addition of amodiaquine to [arbidol+aripiprazole] to create the Triple A combination did not lead to significant enhancement of antiviral activity.

Because the combination of [aripiprazole+arbidol] appeared synergistic by Drug Combination Assay 1, Drug Combination Assay 2 (i.e., checkerboard assay) was performed in additional experiments with these two drugs and the results were analyzed with SynergyFinder2. Several parameters were reported from SynergyFinder2, including the average Bliss Synergy Score of the entire dose-response matrix and the Maximum Synergistic Area (MSA), which corresponds to the maximum Bliss Score calculated over an area of 9 doses of the two compounds in a checkerboard experiment (i.e., 3×3 dose-response matrix). The selective efficacy quantifies the difference between inhibition of virus-infected (Virus) and mock-infected cells (Viability). A selective efficacy of 100 means the drug combination inhibits 100% of virus-infected cells and does not affect mock-infected, drug-treated cells, while a selective efficacy of 0 means the drug combination kills 100% of both virus- and mock-infected cells. While there are no established guidelines on what constitutes actual synergy, recent studies suggest that synergy scores >10 are biologically meaningful. Moreover, an analysis of 448, 555 anticancer drug combination screens (measured across 124 human cancer cell lines) from the DrugCombDB database reveals that among a full spectrum of drug combination effects, the top 5% of most synergistic drug combinations exhibit synergy scores >12. Thus, the threshold for synergy (i.e., synergy scores >10) aligns with available drug combination data. The combination of [aripiprazole+arbidol] conferred synergistic suppression of JUNV and LASV pseudovirus infection, consistent with the FIC and overall Bliss Synergy scores from Drug Combination Assay 1. The MSA scores were 17.42 and 8.18 for JUNV and LASV, respectively, indicating that there are specific concentration windows that led to synergistic antiviral effects. Moreover, the MSAs for JUNV and LASV fall within the top 3% and 11% of Bliss synergy scores of large-scale screens. The selective efficacy of 57.2 and 31.9 for JUNV and LASV pseudoviruses, respectively, indicates strong selective suppression of virus infection but not cell viability.

Arbidol was then evaluated to determine whether it might show synergy when combined with a different approved drug that also acts as a fusion inhibitor. Sertraline was selected because it (i) has previously been shown to be a fusion inhibitor for both EBOV and LASV, (ii) synergizes with other fusion inhibitors (e.g., toremifene) to suppress EBOV, and (iii) inhibits infectious PICV and LASV (see FIG. 2). SynergyFinder2 analyses of checkerboard assays against JUNV and LASV pseudoviruses showed that [arbidol+sertraline] caused modest synergistic suppression of infection with overall Bliss Synergy scores of 6.45 and 3, MSAs of 9.76 and 9.56, and selective efficacies of 69.6 and 60, respectively. The MSAs for JUNV and LASV fall within the top 8% and 9% of the Bliss synergy scores of large-scale screens.

The synergistic antiviral effects of [sertraline+arbidol] prompted evaluation of these two drugs with a third drug, aripiprazole, because [arbidol+aripiprazole] appeared to be the most synergistic two-drug combination in our three-drug screen (see FIGS. 3A-3C and FIGS. 4A and 4B). All two-drug combinations produced FIC scores <1 when tested against LASV and JUNV pseudoviruses (FIG. 5E, panels E and F). The synergistic effects occurred at lower concentrations of each drug in the mixture that were not toxic to cells. For LASV, all two-drug combinations appeared to confer similar degrees of synergistic inhibition, while for JUNV, the combination of [arbidol+sertraline] seemed to be the most potent two-drug combination. Although the triple-drug combination [arbidol+sertraline+aripiprazole] conferred the lowest FIC, the Bliss Synergy Score calculated by SynergyFinder2 for the triple-drug combination was similar to those for the two-drug combinations.

As described herein, the in vitro antiviral action of the clinically used anti-influenza virus drug arbidol is confirmed and expanded against filoviruses including EBOV and MARV and arenaviruses including LASV, JUNV, TACV, LCMV, and PICV. For the first time it is shown that arbidol (a fusion inhibitor), when combined with the approved drugs aripiprazole (a macropinocytosis inhibitor) or sertraline (a fusion inhibitor), synergistically inhibits LASV and JUNV pseudovirus infection. Moreover, arbidol, amodiaquine, aripiprazole, sertraline, and niclosamide suppress infectious PICV while arbidol, sertraline and niclosamide suppress infectious LASV.

The relationship between in vitro IC₅₀ values and the concentrations achieved in human plasma and tissues following oral administration of these drugs was evaluated. The recommended dosage of arbidol for the treatment of influenza is 200 mg orally three to four times daily for five days, and longer durations of treatment up to 20 days have been reported. The same frequency and varying duration are currently in use for COVID-19, and single doses of up to 800 mg have been administered without adverse effects. The maximum plasma concentration (Cₘₐₓ) after a single oral 200-mg dose in humans ranges from 0.9-1.5 μM, which is lower than the in vitro IC₅₀ values described herein when arbidol is given as a monotherapy against arenaviruses (6-10 μM; see FIGS. 1A-1C and 2). For filoviruses, arbidol monotherapy suppresses EBOV and MARV with in vitro IC₅₀s of 2.7-3.9 μM. However, a single 800-mg dose elicits a Cₘₐₓ of 4 μM and with repeated daily dosing over several days, drug accumulation in plasma and tissues infected by viruses may occur. As described herein, combining arbidol with aripiprazole or sertraline reduced the IC₅₀ of arbidol about 2-fold to 3-4 μM (See FIGS. 3A-3C and 5A-5E), a concentration achievable in plasma following oral administration of arbidol. For aripiprazole, Cₘₐₓ in humans ranges from 0.17-1.0 μM upon administration of 5-30 mg daily for 14 days. However, Cₘₐₓ values of 2-5 μM after 14 days have been reported, which are near the in vitro IC₅₀ values for aripiprazole (4.5-6.0 μM) that synergized with arbidol or sertraline to suppress LASV and JUNV infection. For sertraline, Cₘₐₓ ranges from 0.07-0.18 μM after a single 25-100 mg dose, while after 21 days of a 200-mg daily dose, the Cₘₐₓ ranges from 0.39-0.54 μM. Thus, the in vitro IC₅₀ of sertraline (3.6-5.4 μM) that synergized with arbidol or aripiprazole to suppress LASV and JUNV infection is currently higher than what can be achieved in vivo.

Most of the current drug countermeasures for emerging and re-emerging acute viral infections are single agents, yet all successful antiviral therapies for chronic viral infections are based on combinations of drugs. A stockpile of orally available, room temperature stable combinations of approved drugs that synergistically inhibit infections by arenaviruses, filoviruses, and possibly members of other virus families at multiple stages of infection could reduce viral loads, virus-induced inflammation (e.g., cytokine storms), pathogenesis, and case fatality rate. Such drug combinations could provide coverage when new strains arise that are not covered by available vaccines and/or therapeutic antibodies and/or drugs directed against specific viral proteins. Moreover, targeting different stages of infection would reduce the likelihood of emergence of drug resistant strains. Stochastic models have shown that aggressive deployment of antiviral medications can curtail an outbreak, and World Health Organization guidelines state that “unless a country has a stockpile, it will not have antivirals available to use in a pandemic”, the current situation with the SARS-CoV-2 pandemic. Because the development, safety and efficacy testing, scaleup, and deployment of vaccines, therapeutic antibodies, and designer antiviral drugs are cost- and time-intensive, a stockpile of approved oral drugs/drug combinations with activity against related virus family members could be invaluable as a first line of defense to reduce virus transmission and related morbidities and mortalities during the initial waves of pandemic infections.

In summary, arbidol and several other approved drugs inhibit multiple arenaviruses, and when arbidol is combined with other approved drugs, the drug combinations exert synergistic suppression of arenaviruses. As described herein, repurposing combinations of approved oral drugs is a proactive way forward for global preparation as a rapidly deployable, first line of defense for future virus outbreaks and perhaps even for the current SARS-CoV-2 pandemic. In this regard, approved drug screens have shown that the same drugs inhibit EBOV, LASV, SARS and MERS, SARS-CoV-2, and many other viruses. Repurposing of approved drugs in carefully tested and validated combinations may offer a proactive new strategy for controlling known and new viral outbreaks in the future through (i) cost-reductions in antiviral drug development, (ii) application to other medically significant viruses that share similar routes of entry into cells, (iii) enhanced outbreak readiness through stockpiling without a need for cold chain storage, and (iv) affordability for global deployment.

### Materials and Methods

Chemicals, cell culture, and live virus. Vero, Vero E6, and 293T cells were maintained in standard medium (Dulbecco's Modified Eagle Medium (DMEM; Gibco, 11995-065) supplemented with 9% fetal bovine serum (FBS; Gibco, 16000-044) and 1% penicillin-streptomycin (Gibco, 15140-122)). Arbidol (Arb) was synthesized commercially, and the purity and structure of the product were confirmed as described (Pecheur E I, Borisevich V, Halfmann P, Morrey J D, Smee D F, Prichard M, Mire C E, Kawaoka Y, Geisbert T W, Polyak S J. 2016. The Synthetic Antiviral Drug Arbidol Inhibits Globally Prevalent Pathogenic Viruses. J Virol 90:3086-92). Amodiaquine (Amo) was purchased from Sigma Aldrich (A2799-5G); and aripiprazole (Ari) and sertraline were purchased from Selleckchem (S1975 and S4053, respectively). Infectious Pichinde virus (PICV-GFP) was a recombinant virus, rP18tri-GFP, that expresses GFP as a reporter (Dhanwani R, Zhou Y, Huang Q, Verma V, Dileepan M, Ly H, Liang Y. 2015. A Novel Live Pichinde Virus-Based Vaccine Vector Induces Enhanced Humoral and Cellular Immunity after a Booster Dose. J Virol 90:2551-60). The parental virus was generated from a passage 18 of PICV that produces Lassa-like disease in guinea pigs (Aronson J F, Herzog N K, Jerrells T R. 1994. Pathological and virological features of arenavirus disease in guinea pigs. Comparison of two Pichinde virus strains. Am J Pathol 145:228-35). Lassa (Josiah) strain was propagated as described in Saikh K U, Morazzani E M, Piper A E, Bakken R R, Glass P J. 2020. A small molecule inhibitor of MyD88 exhibits broad spectrum antiviral activity by up regulation of type I interferon. Antiviral Res 181:104854.

Production of pseudovirus. Murine leukemia reporter viruses pseudotyped with glycoproteins (GPs) from filoviruses (EBOV (Zaire, Mayinga isolate; GP plasmid), MARV (Angola and Musoke isolates; GP plasmids), and arenaviruses (LASV (Josiah isolate; GP plasmid), JUNV, LCMV (GP plasmid), and PICV (wild type (WT) and R55A mutant (Shao J, Liu X, Ly H, Liang Y. 2016. Characterization of the Glycoprotein Stable Signal Peptide in Mediating Pichinde Virus Replication and Virulence. J Virol 90:10390-10397) were generated as follows. 293T cells were seeded in a T175 flask (Corning; 431080) in transfection medium (DMEM without phenol red (Gibco, 31053036), 9% FBS, 1% L-glutamine (Gibco, 25030-081), 1% sodium pyruvate (Gibco, 11360-070) and incubated at 37° C., 5% CO₂ overnight. The next day, 1000 μL Opti-MEM (Gibco, 11058-021) were added to a 1.5 mL tube, followed by 55.2 μL of X-tremeGENE 9 DNA Transfection Reagent (Roche Applied Science; cat. 06 365 787 001). Plasmids pTG-Luc (7.4 μg), pCMV-MLV (gag-pol; 3.7 μg), gag-BlaM (3.7 μg), and the respective viral fusion GP (3.7 μg) were then added to the tube. After a quick vortex and brief spin, transfection mixtures were incubated for 15 minutes at room temperature followed by another quick vortex and spin. Complexes were then added to the previously seeded 293T T175 flasks and incubated at 37° C., 5% CO₂ for 48 hours. Following incubation, each pseudovirus (PV) preparation was harvested by adding medium from the T175 flask to two 15 mL Falcon tubes (Corning, 352196), followed by centrifugation at 800 g at 4° C. for 7 minutes to pellet cell debris. Aliquots (1 mL) of each PV stock were stored at −80° C., and freeze-thaws were avoided.

Detection of viral glycoproteins. Viral GPs were detected by western blot using an arenavirus-specific monoclonal antibody (22.5D, Zalgen Labs), an Ebola-virus specific monoclonal antibody (H3C8; J Virol Methods. 2011 June; 174(1-2): 99-109. PMID: 21513741), and a rabbit anti-MARV GP polyclonal antibody (catalog #: 0303-007, IBT Bioservices). MLV p30 gag protein was detected with a mouse monoclonal anti-MLV p30 antibody [4B2] (catalog #: ab130757, Abcam). The GPs were detected in non-concentrated pseudovirus stocks. Western blots were performed as described in Polyak S J, Morishima C, Shuhart M C, Wang C C, Liu Y, Lee D Y. 2007. Inhibition of T-cell inflammatory cytokines, hepatocyte NF-kappaB signaling, and HCV infection by standardized Silymarin. Gastroenterology 132:1925-36.

Infection of cells with pseudovirus or live virus. Pseudovirus stocks were thawed and allowed to come to room temperature. Using a multichannel pipette, 100 μL of virus stock were added to each well of columns 2-12 on three 96-well plates. Column 1 on each plate served as the mock infected control: 100 μL of standard medium were added to each well in column 1. Plates were then spun at 300 g for 1 hour at 4° C., after which the plate was taken to a biosafety cabinet and the lid removed for 10-15 seconds to evaporate the condensate on the lid. The plate was then incubated at 37° C., 5% CO₂ for 24 hours. For experiments with arbidol, Vero cells were pretreated with the six to eight different drug concentrations prior to infection of cells with pseudovirus as described previously (Hulseberg C E, Feneant L, Szymanska-de Wijs K M, Kessler N P, Nelson E A, Shoemaker C J, Schmaljohn C S, Polyak S J, White J M. 2019. Arbidol and Other Low-Molecular-Weight Drugs That Inhibit Lassa and Ebola Viruses. J Virol 93). For testing of arbidol against PICV-GFP, Vero E6 cells were pretreated with varying drug concentrations for 1 hour prior to infection with PICV-GFP at a multiplicity of infection (MOI) of 0.1. For LASV Josiah infections, Vero E6 cells were treated with varying concentrations of drugs for 1 hour prior to infection with LASV Josiah at an MOI of 0.2, and a cell-based ELISA was used to quantify viral infectivity (Saikh K U, Morazzani E M, Piper A E, Bakken R R, Glass P J. 2020. A small molecule inhibitor of MyD88 exhibits broad spectrum antiviral activity by up regulation of type I interferon. Antiviral Res 181:104854). For both viruses, all conditions were conducted in triplicate.

Fluorescence microscopy and quantitation for PICV-GFP. At 48 hours post-infection, medium was removed from PICV-GFP-infected Vero E6 cultures, and cells were washed in PBS and fixed in 4% paraformaldehyde for 20 minutes at room temperature. Cells were then washed twice in PBS, then permeabilized in 0.3% Triton-X in PBS for 15 minutes. Fixed cells were then stained with DAPI and imaged using Cytation 1 Cell imaging System (BioTek, Winooski, Vt.) using 10× objective and light cubes for DAPI (nuclear stain) and GFP (infection reporter). Gen5 software (Biotek) was used for image acquisition, processing, and subsequent analysis. The 10× objective was used to take 64 individual images using an 8×8 matrix, which were stitched together utilizing Gen5's montage feature. DAPI threshold was set at 4,000 relative fluorescent units and GFP threshold was set at 900 relative fluorescent units to identify nuclei and infected cells, respectively. Analysis identified nuclei in DAPI channel utilizing a minimum and maximum size selection of 8 μm and 35 μm to capture only stained nuclei. GFP positive cells were identified utilizing minimum and maximum size selection of 15 μm and 58 μm to capture entire GFP positive cells.

Cell viability assays. For approved drug dose-response studies, cell viability was measured in parallel plates using the ATPlite kit (Perkin Elmer, 6016943). For most drug combination assays, PrestoBlue (PB) HS Cell Viability Reagent (Invitrogen, P50201) to measure was used cell viability in the same wells as for virus-produced luciferase measurements. The assay detects the reduction of resazurin to a red-fluorescent dye within the reducing environment of viable cells. This change can be measured by absorbance using 600-nm and 570-nm wavelengths. Briefly, PB was added to wells to 11.1% of well volume and incubated at 37° C., 5% CO₂ for two hours. The plate was then read in a Molecular Devices Spectra max Plus 384 plate reader at 570 nm and 600 nm. The 600-nm readings were subtracted from the 570-nm readings, followed by a subtraction of the average of the medium-only background wells. Replicates were averaged across plates and divided by the average of the solvent+PV control wells to obtain the fraction of control. Results were multiplied by 100 to obtain percent of control. Next, the contents of each well were aspirated, and 50 μL of phosphate buffered saline (PBS; Gibco, 10010031) were added to each well. Fifty μL of Britelite reagent (PerkinElmer, 6066761) were added to each well. The plate was then placed for approximately 10 minutes on low on a plate shaker, then read for luminescence on a Victor plate reader (Perkin Elmer).

Drug Combination Assay 1. The protocol from Cokol-Cakmak et al. (Cokol-Cakmak M, Bakan F, Cetiner S, Cokol M. 2018. Diagonal Method to Measure Synergy Among Any Number of Drugs. J Vis Exp doi:10.3791/57713) was adapted. This method allows three different drugs to be tested individually, in three two-drug combinations, and as a triple combination on a single 96-well plate over a range of ten uniformly divided concentrations. For each set of three drugs, three 96 well plates were used to provide triplicate conditions. Each row of a 96-well plate contained the three single drugs (rows A, B, and C), the three two-drug combinations (rows D, E, and F), and the triple drug combination (row G). The eighth and final row H contained the solvent control, a 1:1 mixture of DMSO:ethanol, at a concentration of 1% in all wells. The final concentration of DMSO and ethanol was 0.5%.

The experimental setup involves 4 steps: (i) defining the stock concentration of each drug, (ii) preparing the drug Master Mix Plate for the seven drug combinations (three single drugs, three two-drug combinations, one three-drug combination) plus one solvent control, (iii) preparing the Drug Dilution Plate, and (iv) transfer of 2 μL of drugs from the Drug Dilution Plate to the three plates of cells. These steps are summarized below. The concentrations of the drugs tested decreased equally by 10% across the dilution series. Thus, single drugs and drug combinations were tested over a concentration range of 0.125-1.25×, with × representing the IC₅₀ of the drug(s) in question. The concentration range was divided in equal intervals of 10% and plated in columns 2 through 11 of the 96-well plate. Specifically, concentrations of 0.125, 0.25, 0.375, 0.5, 0.625, 0.75, 0.875, 1.0, 1.125, and 1.25× the IC₅₀ were tested for the single and drug combinations. For drug combinations, each drug was tested at its separately determined IC₅₀. This method is adaptable in terms of 1) the concentration range tested and 2) how the concentration range is partitioned into 10 equal intervals.

Five thousand Vero cells were seeded into each well of three 96-well, sterile, tissue culture treated, black, clear bottom plates (Perkin Elmer, 6005182) in a final volume of 98 μL of standard medium per well. The three plates were incubated at 37° C., 5% CO₂ overnight. Stock solutions of each drug (A, B, C) were made at 437.5× their previously determined IC₅₀ in 1:1 ethanol:DMSO solvent (200 proof ethanol from Decon Labs, CAS #64-17-5; DMSO from Mediatech, 25-950-CQC), and drug stocks were vortexed thoroughly. Two hundred μL of each drug stock (A, B, C) were added to 500 μL of solvent in separate sterile 1.5-mL Eppendorf tubes to generate 125× stocks of each single drug. To generate stocks of two-drug mixtures, 200 μL of drug A and B (each at 437.5×) were added to 300 μL of solvent in one 1.5-mL tube, yielding a 125× stock of drug A+B. Two-drug mixtures of drug A+C and drugs B+C were similarly generated. To generate the three-drug mixture, 200 μL of drugs A, B, and C (each at 437.5×) were added to 100 μL of solvent in one 1.5-mL tube, yielding a 125× stock of drug A+B+C. The solvent control tube was generated by transferring 700 μL of solvent to a separate 1.5-mL tube. These eight tubes were vortexed, followed by a quick spin. Next, the contents of these eight tubes were separately pipetted into their own well in column 1 in a deep well, sterile, non-tissue culture treated, clear 96-well plate to generate the “Drug Master Mix Plate”. From this plate, for each single, double, or triple drug mixture, 100, 90, 80, 70, 60, 50, 40, 30, 20, 10, and 0 μL was pipetted horizontally (i.e., across the respective row) into each well of columns 2 through 11 of a separate, sterile, clear 96-well plate using a multichannel pipette to yield the “Drug Dilution Plate”. Next, solvent was pipetted across columns 2 through 11 at volumes of 0, 10, 20, 30, 40, 50, 60, 70, 80, 90, and 100 μL. The Drug Dilution Plate therefore represents the concentration range divided into equal intervals as described above, with the final concentrations of the single, double and triple drug mixtures being 12.5, 25, 37.5, 50, 62.5, 75, 87.5, 100, 112.5, and 125×. The lid was placed on the plate, which was placed on a plate shaker at low setting for 10 minutes at room temperature. Using a multichannel pipette, 2 μL from each well on the Drug Dilution Plate were transferred to its corresponding well on one of the three plates of Vero cells plated the day before. This final 1:100 dilution yielded the desired concentration range of 0.125-1.25× divided in 10 equal intervals.

Drug Combination Assay 2. Here, 6×6 checkerboard experiments were performed, (i.e., two drugs were tested at all possible combinations for 36 combinations of two drugs). Five thousand Vero cells were seeded into each well of two 96-well black, clear bottom plates in a final volume of 98 μL of standard medium per well. The two plates were incubated at 37° C., 5% CO₂ overnight. Stock solutions of each drug (A, B) were made at 400× their previously determined IC₅₀ in 1000 μL 1:1 ethanol:DMSO solvent, in separate sterile 1.6-mL Eppendorf tubes, and drug stocks were vortexed thoroughly. Five hundred μL of each drug stock (A, B) were added to 500 μL of solvent in separate sterile 1.6-mL Eppendorf tubes. This step was serially repeated four times to generate five concentrations of each drug, with 2-fold differences between each concentration. A final 1.6-mL Eppendorf tube was filled with 500 μL of solvent. The dilution series for each drug and solvent were added to its own row of a deep well, sterile, non-tissue culture treated, 96-well plate. In a sterile, non-tissue culture treated, 96-well plate Drug Dilution Plate, 100 μL of solvent were pipetted into the first half of the top row (wells A1-A6) of the plate and the first half of the bottom row (wells H1-H6). Fifty μL from the row in the deep well plate containing the serial dilutions of Drug A were pipetted into wells B1-B6 in the drug dilution plate. This procedure was repeated for rows C to G. The drug dilution plate was turned clockwise 90°, and 50 μL from the row in the deep well plate containing the serial dilutions of Drug B were pipetted into columns B1-G1 in the drug dilution plate. This procedure was repeated for columns 2-6 (e.g., B2-G2, B3-G3) to complete the 6×6 checkerboard. The lid was placed on the plate, which was then placed on a plate shaker at low setting for 10 minutes at room temperature. Using a multichannel pipette, 2 μL from each well on the Drug Dilution plate were transferred to the two cell plates. Each 96-well plate allowed plating of two 6×6 checkerboard per plate. Thus, each two-drug concentration tested in the 6×6 checkerboard was performed in quadruplicate.

Data and drug combination analyses. For single drug experiments with arbidol, drug concentrations were log transformed and the concentration of drug(s) that inhibited virus by 50% (i.e., IC₅₀), and the concentration of drug(s) that killed 50% of cells (i.e., CC₅₀), were determined via nonlinear logistic regressions of log(inhibitor) versus response-variable dose-response functions (four parameters) constrained to zero bottom asymptote by statistical analysis using GraphPad Prism 9 (GraphPad Software, Inc.) as described in Hulseberg C E, Feneant L, Szymanska-de Wijs K M, Kessler N P, Nelson E A, Shoemaker C J, Schmaljohn C S, Polyak S J, White J M. 2019. Arbidol and Other Low-Molecular-Weight Drugs That Inhibit Lassa and Ebola Viruses. J Virol 93. The Selectivity Index was calculated by dividing CC₅₀ by the IC₅₀.

For Drug Combination Assay 1, Z-factor was calculated as follows:

$${Z}^{′} = 1 - \frac{3 ( {\hat{σ}}_{p} + {\hat{σ}}_{n} )}{\vert {\hat{μ}}_{p} - {\hat{μ}}_{n} \vert}$$

$$Sample Standard Deviations = \hat{σ} \\ Sample Means = \hat{μ}$$

(p) subscript indicates positive control, (n) subscript indicates negative control. Data presented herein include data from LASV and JUNV pseudovirus infection experiments with average Z-factors of 0.71+/−0.06 and 0.5+/−0.11, respectively.

Fractional Inhibitory Concentration (FIC) scores were calculated by dividing the observed IC₅₀ by the expected IC₅₀ for each two-drug and three-drug combination. The expected IC₅₀ for the two- or three-drug combinations was calculated by the arithmetic mean of the IC₅₀ for each drug tested individually. By examining the single drug dose-response data, the concentration closest to 50% inhibition was designated as the observed IC₅₀. FICs of 1 suggest additivity, FICs >1 suggest antagonism, and FICs <1 suggest synergism. Data from multiple experiments were analyzed by one-way analysis of variance (ANOVA) using Tukey's multiple comparisons test in GraphPad Prism 9.

Data from three-drug combination (Drug Combination Assay 1) and two-drug checkerboard tests (Drug Combination Assay 2) were analyzed in SynergyFinder2, an open-access platform for multi-drug combination synergies (Ianevski A, Giri A K, Aittokallio T. 2020. SynergyFinder 2.0: visual analytics of multi-drug combination synergies. Nucleic Acids Res doi:10.1093/nar/gkaa216; and Ianevski A, He L, Aittokallio T, Tang J. 2017. SynergyFinder: a web application for analyzing drug combination dose-response matrix data. Bioinformatics 33:2413-2415). For the combination synergy model, the Bliss independence model was used, which assumes a stochastic process in which the drugs elicit their effects independently, and the expected combination effect can be calculated based on the probability of independent events. Several parameters were reported from SynergyFinder2, including the average Bliss Synergy Score of the entire dose-response matrix and the Maximum Synergistic Area (MSA), which corresponds to the maximum Bliss Score calculated over an area of 9 doses of the two compounds in a checkerboard experiment (i.e., 3×3 dose-response matrix). The Selective Efficacy was calculated as the average percent viability difference between efficacy (viability of virus-infected cells) and toxicity (viability of control cells). Selective Efficacy quantifies the difference between inhibition of virus-infected (Virus) and mock-infected cells (Viability). A selective efficacy of 100 means that drug combination inhibits 100% of virus-infected cells and does not affect mock-infected cells, while selective efficacy of 0 means that drug combination inhibits 100% of both virus- and mock-infected cells.

While illustrative embodiments have been illustrated and described, it will be appreciated that various changes can be made therein without departing from the spirit and scope of the invention.

1. A method for inhibiting or treating viral infection in a subject infected with a virus of the arenaviridae family, comprising administering to a subject in need thereof a therapeutically effective amount of a therapeutic agent combination comprising arbidol, or a pharmaceutically acceptable salt thereof, and a second therapeutic agent or a combination of second therapeutic agents selected from the group consisting of:
(a) aripiprazole or a pharmaceutically acceptable salt thereof;
(b) amodiaquine or a pharmaceutically acceptable salt thereof;
(c) sertraline or a pharmaceutically acceptable salt thereof;
(d) aripiprazole and amodiaquine, or pharmaceutically acceptable salts thereof; and
(e) aripiprazole and sertraline, or pharmaceutically acceptable salts thereof.

2. The method of claim 1, comprising administering to a subject in need thereof a therapeutically effective amount of a therapeutic agent combination selected from the group consisting of:
(a) arbidol and aripiprazole, or pharmaceutically acceptable salts thereof;
(b) arbidol and amodiaquine, or pharmaceutically acceptable salts thereof;
(c) arbidol and sertraline, or pharmaceutically acceptable salts thereof;
(d) arbidol, aripiprazole, and amodiaquine, or pharmaceutically acceptable salts thereof; and
(e) arbidol, aripiprazole, and sertraline, or pharmaceutically acceptable salts thereof.

3. The method of claim 1, comprising administering to a subject in need thereof a therapeutically effective amount of a therapeutic agent combination comprising arbidol and aripiprazole, or pharmaceutically acceptable salts thereof.

4. The method of claim 1, comprising administering to a subject in need thereof a therapeutically effective amount of a therapeutic agent combination comprising arbidol and amodiaquine, or pharmaceutically acceptable salts thereof.

5. The method of claim 1, comprising administering to a subject in need thereof a therapeutically effective amount of a therapeutic agent combination comprising arbidol and sertaline, or pharmaceutically acceptable salts thereof.

6. The method of claim 1, comprising administering to a subject in need thereof a therapeutically effective amount of a therapeutic agent combination comprising arbidol, aripiprazole, and amodiaquine, or pharmaceutically acceptable salts thereof.

7. The method of claim 1, comprising administering to a subject in need thereof a therapeutically effective amount of a therapeutic agent combination comprising arbidol, aripiprazole, and sertraline, or pharmaceutically acceptable salts thereof.

8. A method for inhibiting or treating viral infection in a subject infected with a virus of the arenaviridae family, comprising administering to a subject in need thereof a therapeutically effective amount of a therapeutic agent combination comprising aripiprazole and amodiaquine, or pharmaceutically acceptable salts thereof.

9. The method of claim 1, wherein the arenavirus is an Old World Arenavirus.

10. The method of claim 1, wherein the arenavirus is a New World Arenavirus.

11. The method of claim 1, wherein the arenavirus is Lassa virus, Junin virus, Guanarito virus, Lujo virus, Machupo virus, Sabia virus, or Whitewater Arroyo virus.

12. (canceled)

13. The method of claim 1, wherein the therapeutic agent combination is administered as a single composition.

14. (canceled)

15. The method of claim 1, wherein the therapeutic agent combination is orally administered.

16. The pharmaceutical composition of claim 25 comprising arbidol and aripiprazole, or pharmaceutically acceptable salts thereof.

17. The pharmaceutical composition of claim 25 comprising arbidol and amodiaquine, or pharmaceutically acceptable salts thereof.

18. The pharmaceutical composition of claim 25 comprising arbidol and sertraline, or pharmaceutically acceptable salts thereof.

19. The pharmaceutical composition of claim 25 comprising aripiprazole and amodiaquine, or pharmaceutically acceptable salts thereof.

20. The pharmaceutical composition of claim 25 comprising arbidol, aripiprazole, and amodiaquine, or pharmaceutically acceptable salts thereof.

21. The pharmaceutical composition of claim 25 comprising arbidol, aripiprazole, and sertraline, or pharmaceutically acceptable salts thereof.

22-24. (canceled)

25. A pharmaceutical composition comprising a therapeutic agent combination selected from the group consisting of:
(a) arbidol and aripiprazole, or pharmaceutically acceptable salts thereof;
(b) arbidol and amodiaquine, or pharmaceutically acceptable salts thereof;
(c) arbidol and sertraline, or pharmaceutically acceptable salts thereof;
(d) arbidol, aripiprazole, and amodiaquine, or pharmaceutically acceptable salts thereof;
(e) arbidol, aripiprazole, and sertraline, or pharmaceutically acceptable salts thereof; and
(f) aripiprazole and amodiaquine, or pharmaceutically acceptable salts thereof.
